(19) United States
(12) Patent Application Publication
(10) Pub. No.: US 2003/0162195 A1
(43) Pub. Date: Aug. 28, 2003

(54) Prediction of cancer by detection of ATM mutations

(76) Inventors: Shlomit Gilad, Gedera (IL); Joachim Yahalom, New York, NY (US)

Correspondence Address: John P. White, Cooper & Dunham LLP, 1185 Avenue of the Americas, New York, NY, 10036, US

(21) Appl. No.: 10/251,210

(22) Filed: Sep. 20, 2002

Related U.S. Application Data
(60) Provisional application No. 60/323,766, filed on Sep. 20, 2001.

(51) Int. Cl. C12Q 1/68

(52) U.S. Cl. 435/6

(57) ABSTRACT

There is provided a method of testing a subject to determine if the subject has a predisposition for developing a cancer, a cancer of epithelial origin such as lung cancer, colon cancer, prostate cancer, ovarian cancer, bladder cancer, and cancer of the pancreas, and also a lymphoproliferative malignancy such as Hodgkin's disease and non-Hodgkin's lymphoma. This method includes the steps of detecting a mutation in the open reading frame of the ATM gene (SEQ.ID.NO:1) in a cDNA sample or a genomic DNA sample from the subject, which mutation is selected from the group consisting of the mutations set forth in Table 3 and Table 4; or, detecting a mutation in the mRNA corresponding to the open reading frame of the ATM gene (SEQ.ID.NO:1) in a mRNA sample from the subject, which mutation is selected from the group consisting of RNA complementary to the mutations set forth in Table 3 and Table 4, wherein the presence of such a mutation indicates that the subject has a predisposition for developing cancer. Also provided is an isolated cDNA molecule having a nucleotide sequence which differs from the sequence set forth in SEQ.ID.NO:1 by a mutation selected from the group consisting of mutations 378 T→A, 3383 A→G, 1636 C→G, 2614 C→T, 6437 G→C, 2932 T→C, 2289 T→A, 6096 A→T, 6176 C→T, 6919 C→T, 2442 C→A, 3925 G→A, 6067 G→A, 2119 T→C, 1810 C→T, and 4388 T→G. An oligonucleotide probe which is capable of detecting a mutation in the open reading frame of the ATM gene is also provided. Additionally, kits for detection and prediction of cancer are provided.

This invention is a continuation-in-part and claims the benefit of U.S. Provisional Application No. 60/323,766, filed Sep. 20, 2001, the contents of which are hereby incorporated by reference into this application.

### BACKGROUND OF THE INVENTION

1. Field of the Invention

The present invention generally relates to the relationship of ATM germine point mutations to cancer, in particular cancers of epithelial origin such as lung cancer, colon cancer, prostate cancer, ovarian cancer, bladder cancer, and cancer of the pancreas. The present invention also relates to the relationship of an ATM germline point mutation to a lymphoproliferative malignancy such as Hodgkin's disease and non-Hodgkin's lymphoma. More specifically, the present invention relates to the use of this relationship in prediction and detection of cancers prior to large tumor growth.

2. Description of Related Art

Ataxia Telangiectasia (A-T) is a pleiotrophic inherited disease characterized by neurodegeneration, cancer, immunodeficiencies, radiation sensitivity, and genetic instability. The gene, a mutation in which is responsible for A-T, is called ATM, and it was discovered by Shiloh et al. in 1995 (Savitsky, K et al., 1995). The ATM gene extends over 150 kb of genomic DNA (Uziel, T et al., 1996) and is transcribed into a large transcript of about 13 kb, representing 66 exons (Uziel, T et al., 1996; Savitsky, K et al., 1995; Savitsky, K et al., 1997). The open reading frame of this transcript predicts a 370 kDa protein composed of 3,056 amino acids. The ATM product is homologous to several cell cycle checkpoint proteins from other organisms and is thought to play a crucial role in a signal transduction network that modulates cell cycle checkpoints, genetic recombination, apoptosis and other cellular responses to DNA damage (Meyn, MS, 1999).

A-T cells respond abnormally to radiation-induced DNA damage and are remarkably sensitive to ionizing radiation. M. Swift and others (Morrel, D, et al. 1990; Swift, M, et al., 1987; Swift, M, et al., 1991; Easton, D F, 1994) have suggested that exposure to radiation may predispose A-T carriers (heterozygotes) to the development of cancer more than non-carriers (Morrel, D, et al. 1990; Swift, M, et al., 1987; Swift, M, et al., 1991; Easton, D F, 1994). Studies of relatives of A-T patients have provided consistent support for the proposed increased risk of breast cancer in female A-T heterozygotes. (Meyn, MS, 1999). Thus, there is compelling evidence that the ATM gene may play a role in at least one type of cancer (Morrel, D et al. 1990; Swift, M et al., 1987; Swift, M et al., 1991; Easton, D F 1994; PCT Patent Application, Publication No. WO 01/68668 (Skaliter, R and Gilad, S), published 20 Sep. 2001).

In addition, several studies have shown an increased risk for the development of breast cancer in women who had previously been treated with radiotherapy for Hodgkin's Disease (HD) (Hancock, S L, et al., 1993; Yahalom, J, et al., 1992; Aisenberg, A C, et al., 1997).

### SUMMARY OF THE INVENTION

According to the present invention, there is provided a method of testing a subject to determine if the subject has a predisposition for developing a cancer, in particular a cancer of epithelial origin such as lung cancer, colon cancer, prostate cancer, ovarian cancer, bladder cancer, and cancer of the pancreas, and also a lymphoproliferative malignancy such as HD and non-Hodgkin's lymphoma.

This method includes the steps of detecting a mutation in the open reading frame of the ATM gene (SEQ.ID.NO:1) in a cDNA or in a genomic DNA sample from the subject, which mutation is selected from the group consisting of the mutations set forth in Table 3 and Table 4, or detecting a mutation in the mRNA corresponding to the open reading frame of the ATM gene (SEQ.ID.NO:1) in a mRNA sample from the subject, which mutation is selected from the group consisting of RNA complementary to the mutations set forth in Table 3 and Table 4, wherein the presence of such mutation indicates that the subject has a predisposition for developing a cancer, especially a cancer of epithelial origin, or a lymphoproliferative malignancy

Also provided is an isolated cDNA molecule and an isolated genomic DNA molecule having a nucleotide sequence that differs from the sequence set forth in SEQ.ID.NO:1 by a mutation selected from the group consisting of mutations 378 T→A, 3383 A→G, 1636 C→G, 2614 C→T, 6437 G→C, 2932 T→C, 2289 T→A, 6096 A→T, 6176 C→T, 6919 C→T, 3925 G→A, 6067 G→A, 2119 T→C, 1810 C→T, and 4388 T→G. A marker for determining a predisposition for cancer, especially the above-referenced cancers, is also provided. Kits are also provided for detecting mutations in the ATM gene.

### DESCRIPTION OF THE DRAWING

Other advantages of the present invention can be readily appreciated, as the same becomes better understood by reference to the following detailed description when considered in connection with the accompanying drawing wherein:

FIG. 1 shows the complete open reading frame (ORF) sequence of the ATM gene (SEQ.ID.NO1), wherein the first codon is ATG (Met) and the last codon is the stop codon (TGA). The entire transcript can be found under GenBank Accession No. U33841, and all of the designations of ATM mutations herein refer to this sequence.

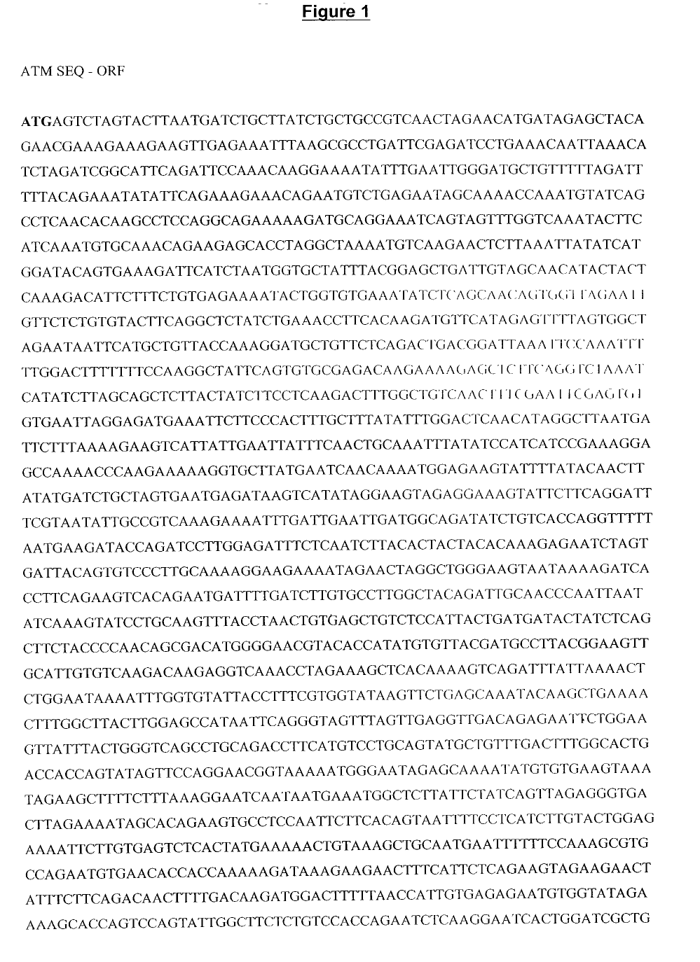

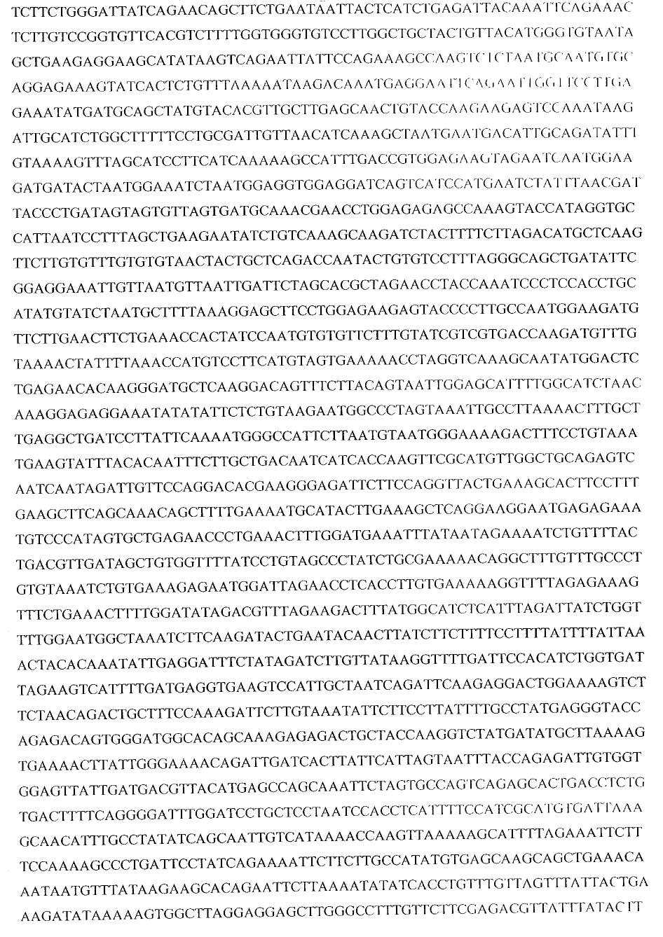

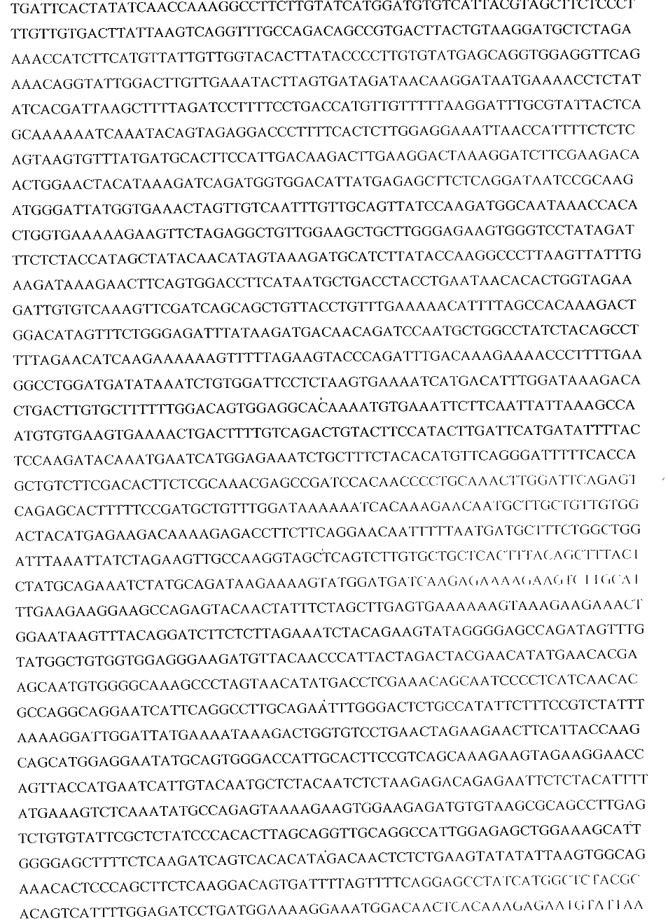

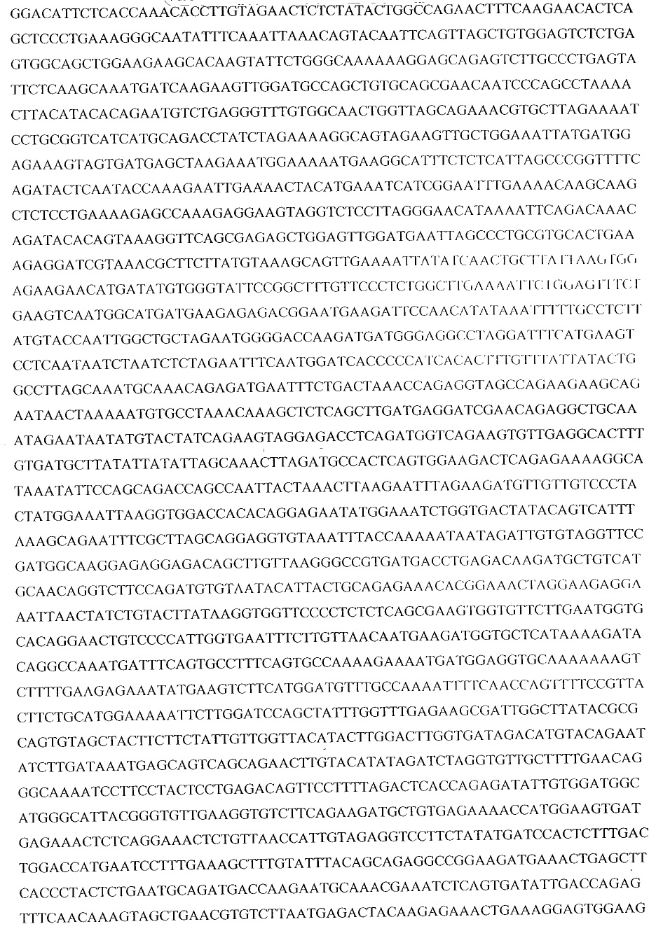

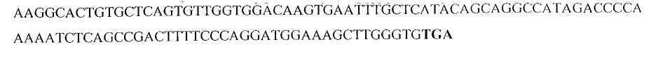

### DETAILED DESCRIPTION OF THE INVENTION

Generally, the present invention provides a method of testing a subject to determine if the subject has a predisposition for developing a cancer, in particular a cancer of epithelial origin (known as a carcinoma) such as lung cancer, colon cancer, prostate cancer, ovarian cancer, bladder cancer, and cancer of the pancreas, and also a lymphoproliferative malignancy such as Hodgkin's disease (HD) and non-Hodgkin's lymphoma.

The methods of the present invention provide that either healthy men and women and/or men and women at risk for suffering from a cancer are screened by obtaining various patient-derived materials such as tissue samples or blood, preferably blood, which is then examined by methods known in the art for the presence of one or more mutation. The tissue samples can include, but are not limited to, blood, mouth brush secretions, other secretions and other tissues.

The methods which are used to detect the presence of the mutations include, but are not limited to, the methods discussed below. There are many methods known in the art for testing DNA for mutations, including point mutations, as described in this specification. Methods that can be used for testing the mutations require use of the primers described in the specification. Mutation detection methods that are used can include, but are not limited to, polymerase chain reaction (PCR)-restriction enzyme assay (Sueoka, H et al., 2000), PCR and LightCycler technology (Funayo, T et al., 2000; Pais, G et al., 2001), allele-specific PCR (MacLeod, S L et al., 2000), restriction enzyme digestion (Ho, L L et al., 2001), denaturing high performance liquid chromatography (dHPLC) for fast and sensitive analysis of PCR-amplified DNA fragments (Oldenburg, J et al., 2001), restriction endonuclease fingerprinting single-strand conformation polymorphism (REF-SSCP) (Jugessur, A et al., 2000; Liu, Q et al., 1995), and detection of single base substitutions as heteroduplex polymorphisms (White, B M et al., 1991).

More specifically, the method of the present invention includes the steps of detecting a mutation in the open reading frame of the ATM gene (SEQ.ID.NO:1) in a cDNA sample from the subject, wherein the mutation is selected from the group consisting of the mutations set forth in Table 3 and Table 4, wherein the presence of such a mutation indicates that the subject has a predisposition for developing a cancer, especially a cancer of epithelial origin such as lung cancer, colon cancer, prostate cancer, ovarian cancer, bladder cancer, and cancer of the pancreas, and also a lymphoproliferative malignancy such as HD and non-Hodgkin's lymphoma.

In another embodiment of the present invention, the method of the present invention can include the step of detecting a mutation corresponding to a mutation in the open reading frame (ATM transcript) of the ATM gene (SEQ.ID.NO:1) in a genomic DNA sample from the subject, wherein the mutation is selected from the group consisting of the mutations set forth in Table 3 and Table 4, wherein the presence of such a mutation indicates that the subject has a predisposition for developing a cancer, especially a cancer of epithelial origin such as lung cancer, colon cancer, prostate cancer, ovarian cancer, bladder cancer, and cancer of the pancreas, and also a lymphoproliferative malignancy such as HD and non-Hodgkin's lymphoma.

Also provided is an isolated cDNA molecule having a nucleotide sequence which differs from the sequence set forth in SEQ.ID.NO:1 by a mutation selected from the group consisting of mutations in position 378 T→A, position 3383 A→G, position 1636 C→G, position 2614 C→T, position 6437 G→C, position 2932 T→C, position 2289 T→A, position 6096 A→T, position 6176 C→T, position 6919 C→T, position 2442 C→A, position 3925 G→A, position 6067 G→A, position 2119 T→C, position 1810 C→T, and position 4388 T→G.

The presence of at least one of the above mutations in the cDNA molecule is indicative of a predisposition for developing a cancer. Thus, an isolated cDNA molecule having at least one of the above mutations can also be used as a marker for determining a predisposition for a cancer. Therefore, the methods of the present invention enable the practitioner to determine the presence of these mutations prior to the occurrence of a cancer The methods also enable the practitioner to determine whether there is a predisposition for a cancer prior to the occurrence of the cancer in an individual, in particular for a cancer of epithelial origin such as lung cancer, colon cancer, prostate cancer, ovarian cancer, bladder cancer, and cancer of the pancreas, and also for a lymphoproliferative malignancy such as HD and non-Hodgkin's lymphoma.

Thus the above methods are predictive of one or more of the above cancers.

The above discussion provides a factual basis for the use of the marker and method of the present invention. The methods used with, and the utility of, the present invention can be shown by the following non-limiting examples and accompanying figure.

### EXAMPLES

Methods

General methods in molecular biology: Standard molecular biology techniques known in the art and not specifically described were generally followed as in Sambrook et al., Molecular Cloning: A Laboratory Manual, Cold Spring Harbor Laboratory Press, New York (1989), and in Ausubel et al., Current Protocols in Molecular Biology, John Wiley and Sons, Baltimore, Md. (1989) and in Perbal, A Practical Guide to Molecular Cloning, John Wiley & Sons, New York (1988), and in Watson et al., Recombinant DNA, Scientific American Books, New York and in Birren et al. (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) was carried out generally as in PCR Protocols: A Guide to Methods and Applications, Academic Press, San Diego, Calif. (1990). In-situ (in-cell) PCR, in combination with Flow Cytometry, can be used for detection of cells containing specific DNA and mRNA sequences (Testoni et al., 1996, Blood 87:3822).

### Example 1

### Identification of Mutations Predictive of Breast Cancer

The current experiment was designed to determine whether germline (inherited) sequence variations in ATM influence:

1. breast cancer risk,

2. bilateral breast cancer risk, and/or

3. response to radiation therapy.

The study populations were composed of three groups:

1. contralateral breast cancer patients (BC-BC) (with or without irradiation treatment),

2. primary breast cancer patients, and

3. age-matched healthy women.

The strategy for identification of the mutations was based on sequencing the entire cDNA. Confirmation of the mutations identified on the cDNA was obtained by analysis of the corresponding genomic DNA region. This full sequencing strategy is the best procedure for identifying all types of mutations and is disclosed more fully herein.

Materials and Methods

Total RNA Isolation from Blood Samples

Isolation of total RNA from peripheral blood was performed by Tri Reagent BD (MRC, INC), according to the manufacturer's protocol. Analysis of RNA quality and quantity was performed using the procedures of OD verification and agarose gel electrophoresis.

Reverse Transcription

First strand cDNA was prepared from 2 μg of total RNA. The RNA in a final volume of 5 ml was heated to 85° C. for two minutes and then stored on ice for another two minutes. A mixture comprising 2 μl of 5×Buffer (GibcoBRL), 0.5 μl of 0.5 mg/ml Oligo dT15 (Boehringer), 1 μl of 0.1M DTT (GibcoBRL), 0.5 μl of 10 mM dNTP (Boehringer) and 0.5 μl of RNAsin (Promega) was added and the combination was heated to 42° C. After five minutes of incubation at 42° C., 0.5 μl of Superscript II (GibcoBRL) was added After a further one hour of incubation at 42° C., the entire mixture was heated to 85° C. for two minutes

ATM PCR

Amplification of the 9355bp ATM transcript was carried out with the primers ATMF and ATMR (Table 1) in a final volume of 50 μl, including 1 μl of the RT product, 1 μl of 0.1 mg/ml BSA (BioLabs), 1 μl of 25 pM of each primer, 5 μl of 10×buffer 3 (Boehringer), 2.5 μl of 10 mM dNTP (Boehringer), 0.75 μl of Expand Long Template (Boehringer) and 0.1 μl of Anti-Taq (Chimerx). The amplification was performed in the PE Cycler GeneAmp PCR 9700. The first step comprised heating at 93° C. for five minutes, followed by 20 cycles of 93° C. for 30 seconds alternating with 68° C. for nine minutes (extension) The last step comprised ten cycles beginning as before with 93° C. for 30 seconds alternating with 68° C. for nine minutes, but increasing the 68° C. incubation in each cycle by ten seconds.

RA and RB PCR

Two overlapping fragments, RA (4964 bp) and RB (5062 bp), were amplified using the product of the ATM RT-PCR as template (Table 1). The same mixture described above for the ATM PCR was used for PCR of each of the two fragments, respectively. The amplifications were carried out under the same conditions, except for the extension time, which was 3.30 minutes.

Sequencing

The RA and RB fragments were purified using the QIAGEN PCR purification kit, and 200 ng of each fragment were sequenced with Big Dyes, PE ABI Prism 377, with primers as described in Table 1.

Sample Analysis

For analysis of the chromatograms, the Sequencher (Gene Code Co.) software was used.

Confirmation of Mutations

For the confirmation of each mutation, amplification of the genomic DNA was performed and the relevant region was sequenced.

Control Samples

Genomic DNA of the control samples was amplified and checked, as shown in Table 2.

TABLE 1
Primers used in the study

| | | |
|---|---|---|
| ATM CDNA: | ATMF | GTTGATACTACTTTGACCTTCCGAGTGCA |
| | | GT |
| | ATMR | AGGCTGAATGAAAGGGTAATTCATATACT |
| | | GAAGA |
| ATM RA: | ATMin | GTGCAGTGAGGCATACATCAC |
| | AR | CCTTCAAGTCTTGTCAATGGAAGTGCAT |
| ATM RB: | 2xx | GCCGTGACTTACTGTAAGGATG |
| | ATMout | AAGGCTGAATGAAAGGGTAATTC |
| PRIMERS FOR | LA | GTTGCTGAGATATTTCACA |
| SEQUENCING | 8P | GTTTTGGCTCCTTTCGGATGATG |
| ATM RA: | 8X | CTTAGCAGCTCTTACTATCTTCC |
| | 8K | GAAGATACCAGATCCTTGGAG |
| | 6K | CTGATAATCCCAGAAGACAGCG |
| | 6Q | GAGAATGTGGTATAGAAAAGCAOC |
| | 7out | TTCCTCTCCTTTGTTAGATGCC |
| | 6in | CTAGGTCAAAGCAATATGGACTC |
| | 6F | CCATAGTGCTGAGAACCCTG |
| | 2A | CAGTAATAAACTAACAAACAGGTG |
| | 2P | GCCATATGTGAGCAAGCAG |
| | 2xx | GCCGTGACTTACTGTAAGGATG |
| PRIMERS FOR | 2C | GAGGACCCTTTTCACTCTTGG |
| SEQUENCING | 1JJ | CTGGACATAGTTTCTGGGAGAT |
| ATM RB: | 1C | GTCAGAGCACTTTTTCCGATGC |
| | 3Q | CAATGTGGGGCAAAGCCCTAG |
| | 3D | CAGGATTTTCTAAGCACGTTTCTG |
| | 5F | CCAGAATTTTCAAGCCAGAGGG |
| | 5C | CTGAGTGGCATCTAAGTTTGC |
| | 4F | CCTCTTCCTAGTTTCCGTGTTTC |
| | 4B | CGTGATGACCTGAGACAAGATG |
| | 4A | GAGCAGTCAGCAGAACTTGTAC |

TABLE 2

Confirmation of the mutations in genomic DNA

| | | | | |
|---|---|---|---|---|
| | | | | Expected PCR |
| | | Primers for | | product size |
| | | PCR (see | | (in genomic |
| Number | Mutation* | TABLE 2b) | Mutation region | DNA) |

| | | | | | | | |
|---|---|---|---|---|---|---|---|
| 1 | 3161 | C→G | 6in | + 6B | GAGGCTGATCCTTATTCAAAA | 1.3 | Kb |
| 2 | 2572 | T→C | 6An | + FRn | CATGAATCTATTTAACGATTA | 150 | bp |
| 3 | 6235 | G→A | 3Q | + 3I | TATTCTTTCCGTCTATTTAAAAG | 1.5 | Kb |
| 4 | 3118 | A→G | 6in | + 6B | CTCTGTAAGAATGGCCCTAGT | 1.3 | Kb |
| 5 | 378 | T→A | Uain | + 8qout | ATATCATGGATACAGTGAAAG | 160 | bp |
| 6 | 146 | C→G | 8C | + 8G | CATTCAGATTCCAAACAAGGA | 1.5 | Kb |
| 7 | 5557 | G→A | 1T | + 1X | TTTTACTCCAAGATACAAATGAA | 2.0 | Kb |
| 8 | 1636 | C→G | FJ | + FD | GACTTTGGCACTGACCACCAG | 190 | bp |
| 9 | 2614 | C→T | 6A | + FB | TGCAAACGAACCTGGAGAGAG | 140 | bp |

*The nucleotide number refers to the cDNA ATM sequence. The first nucleotide of ATG of the open reading frame was designated +1.

TABLE 2b

List of the primer sequences

| | |
|---|---|
| Primer | Sequence |

| | |
|---|---|
| 6in | CTAGGTCAAAGCAATATGGACTC |
| 6B | CAGCAAGAAATTGTGTAAATACTTC |
| 6An | GCCATTTGACCGTGGAGAAGTAG |
| FRn | GGTACTTTGGCTCTCTCCAGG |
| 3Q | CAATGTGGGGCAAAGCCCTAG |
| 3I | CGGAAGTGCAATGGTCCCACTG |
| Uain | GCACCTAGGCTAAAATGTCAAG |
| 8qout | ACCACTGTTGCTGAGATATTTC |
| 8C | CCTGATTCGAGATCCTGAAAC |
| 8G | GCATCTTTTTCTGCCTGGAGG |
| 1x | CCCTTTTGAAGGCCTGGATG |
| 1T | GAATCCAAGTTTGCAGGGGTT |
| FJ | GCAGTATGCTGTTTGACTTTGG |
| FD | GAAGAATTGGAGGCACTTCTGTG |
| 6A | CATTTGACCGTGGAGAAGTAGAAT |
| FB | GGTACTTTGGCTCTCTCCAGGT |

TABLE 3
ATM sequence variations in BC/BC patients

| Patient | Nucleotide | Nucleotide | Codon | |
|---|---|---|---|---|
| No. | No. | substitution | No. | Amino-acid substitution |
| #56 | 2572 | T/C | 858 | Phe → Leu |
| | 3161 | C/G | 1054 | Pro → Arg |
| #57 | 5557 | G/A | 1853 | Asp → Asn |
| | 6235 | G/A | 2079 | Val → Ile |
| #61 | 5557 | G/A | 1853 | Asp → Asn |
| | 5558 | A/T | 1853 | Asp → Val |
| #67 | 5557 | G/A | 1853 | Asp → Asn |
| #72 | 5557 | G/A | 1853 | Asp → Asn |
| #73 | 5557 | G/A | 1853 | Asp → Asn |
| | 6007 | | 2002 | Del89 |
| #75 | 3383 | A/G | 1128 | Gln → Arg |
| #80 | 2572 | T/C | 858 | Phe → Leu |
| | 3161 | C/G | 1054 | Pro → Arg |
| #83 | 5557 | G/A | 1852 | Asp → Asn |
| #90 | 5557 | G/A | 1852 | Asp → Asn |
| #93 | 1636 | C/G | 546 | Leu → Val |
| | 2614 | C/T | 872 | Pro → Ser |
| | 6995 | T/C | 2332 | Leu → Pro |
| #95 | 544 | G/C | 182 | Val → Leu |
| | 3118 | A/G | 1040 | Met → Val |
| #97 | 3161 | C/G | 1054 | Pro → Arg |
| #98 | 5557 | G/A | 1852 | Asp → Asn |
| #101 | 5557 | G/A | 1852 | Asp → Asn |
| #102 | 5557 | G/A | 1852 | Asp → Asn |
| #103 | 6235 | G/A | 2079 | Val → Ile |
| | 378 | T/A | 126 | Asp → Glu |
| #107 | 5557 | G/A | 1852 | Asp → Asn |
| | 146 | C/G | 49 | Ser → Cys |
| #112 | 6235 | G/A | 2079 | Val → Ile |
| | 378 | T/A | 126 | Asp → Glu |
| | 6437 | G/C | 2146 | Ser → Thr |
| #114 | 2932 | T/C | 978 | Ser → Pro |
| #121 | 3118 | A/G | 1040 | Met → Val |
| #122 | 3161 | C/G | 1053 | Pro → Arg |
| #124 | 146 | C/G | 49 | Ser → Cys |
| #117 | 2289 | T/A | 763 | Phe → Leu |
| #125 | 5557 | G/A | 1852 | Asp → Asn |
| #131 | 2572 | T/C | 858 | Phe → Leu |
| | 3161 | C/G | 1053 | Pro → Arg |
| #137 | 6176 | C/T | 2059 | Thr → Ile |
| | 6096 | A/T | | Arg → Ser |
| #138 | 4258 | C/T | 1420 | Leu → Phe |
| | 2119 | T/C | 707 | Ser → Pro |

Total: 28 carriers out of 70 patients (40%)
Total carriers minus 5557 G/A mutation carriers: 19 carriers out of 70 patients (27%)

TABLE 4
Mutations found in the cohort of BC-BC patients.

| | | | | | MSKO | | % | % | |
|---|---|---|---|---|---|---|---|---|---|
| | | BC- | MSKO | Healthy | Healthy | % BC- | MSKO | Healthy | |
| No. | Mutation | BC | primary BC | Controls | controls | BC | pri-BC | Controls | Ref |
| 1 | 5557 G→A | 8/70 | 18/76 | 8/63 | | 11.1% | 23.7% | 12.7% | Sandoval, N. |
| | | | | | | | | | et al., 1999 |
| 2 | 3161 C→G | 5/70 | 5/94 | 1/63 | 7/280 | 6.9% | 5.3% | 1.6% | Vorechovsky, |
| | | | | | (2.5%) | | | | I et al, 1996 |
| 3 | 2572 T→C | 3/70 | 2/87 | 0/63 | 2/280 | 4.2% | 2.3% | 0.0% | Vorechovsky, |
| | | | | | (0.7%) | | | | I et al, 1996 |
| 4 | 6235 G→A | 3/70 | 0/54 | 0/63 | 4/288 | 4.2% | 0.0% | 0.0% | Vorechovsky, |
| | | | | | (1.4%) | | | | I et al, 1996 |
| 5 | 3118 A→G | 2/70 | 1/93 | 0/63 | | 2.8% | 1.1% | 0.0% | Vorechovsky, |
| | | | | | | | | | I et al, 1997 |
| 6 | 146 C→G | 2/70 | 5/71 | 0/63 | | 2.8% | 7.0% | 0.0% | Izatt, L., et |
| | | | | | | | | | al., 2000 |
| 7 | 378 T→A | 2/70 | 2/90 | 1/63 | | 2.8% | 2.2% | 1.6% | New |
| 8 | 5558 A→T | 1/70 | 0/75 | 0/63 | 4/268 | 1.4% | 0.0% | 0.0% | Sandoval, N. |
| | | | | | (1.5%) | | | | et al., 1999 |
| 9 | 3383 A→G | 1/70 | 0/89 | 0/63 | | 1.4% | 0.0% | 0.0% | New |
| 10 | 1636 C→G | 1/70 | 10/76 | 0/63 | | 1.4% | 13.2% | 0.0% | New |
| 11 | 2614 C→T | 1/70 | 3/93 | 0/63 | | 1.4% | 3.2% | 0.0% | New |
| 12 | 544 G→C | 1/70 | 0/64 | 0/63 | | 1.4% | 0.0% | 0.0% | Izatt, L., et |
| | | | | | | | | | al., 2000 |
| 13 | 6437 G→C | 1/70 | 0/65 | 0/63 | | 1.4% | 0.0% | 0.0% | New |
| 14 | 2932 T→C | 1/70 | 0/92 | 0/63 | | 1.4% | 0.0% | 0.0% | New |
| 15 | 2289 T→A | 1/70 | 0/85 | 0/63 | 2/246 | 1.4% | 0.0% | 0.0% | New |
| | | | | | (0.8%) | | | | |
| 16 | 2119 T→C | 2/70 | | 2/63 | 2/262 | 2.8% | | 3.2% | Izatt, I., et |
| | | | | | (0.8%) | | | | al, 2000 |
| 17 | 6096 A→T | 1/70 | | 1/63 | | 1.4% | | 1.6% | New |
| 18 | 6176 C→T | 1/70 | | 0/63 | | 1.4% | | 0.0% | New |
| 19 | 4258 C→T | 1/70 | | 2/63 | 1/238 | 1.4% | | 3.2% | Vorechovsky, |
| | | | | | | | | | I. et al., |
| | | | | | | | | | 1996 |

Nine of the mutations that were found in the group of BC-BC patients (Table 4) are new and are herein found, together with the previously known mutations, to be linked to increased risk of breast cancer, i.e., they have not previously been reported to be predictive of breast cancer.

TABLE 5
Sequence variations in healthy controls

| Control | Nucleotide | Nucleotide | Amino Acid | Amino-acid | |
|---|---|---|---|---|---|
| No. | No. | substitution | position | substitution | Reference |
| #2 | 6919 | C→T | 2307 | Leu→Phe | New |
| #6 | 5557 | G→A | 1853 | Asp→Asn | Sandoval, N. et al, 1999 |
| #21 | 5557 | G→A | 1853 | Asp→Asn | Sandoval, N et al, 1999 |
| #26 | 378 | T→A | 126 | Asp→Glu | New |
| | 2442 | C→A | 814 | Asp→Glu | New |
| #29 | 6919 | C→T | 2307 | Leu→Phe | New |
| | 5557 | G→A | 1853 | Asp→Asn | Sandoval, N. et al,. 1999 |
| #36 | 3161 | C→G | 1054 | Pro→Arg | Vorechovsky, I. et al., 1996 |
| | | | | | and Sandoval, N. et al., |
| | | | | | 1999 |
| | 3925 | G→A | 1309 | Ala→Thr | New |
| | 5557 | G→A | 1853 | Asp→Asn | Sandoval, N. et al., 1999 |
| #37 | 5557 | G→A | 1853 | Asp→Asn | Sandoval, N. et al., 1999 |
| #40 | 5557 | G→A | 1853 | Asp→Asn | Sandoval, N. et al., 1999 |
| #42 | 4258 | C→T | 1420 | Leu→Phe | Vorechovsky, I. et al., 1996 |
| | 6067 | G→A | 2023 | Gly→Arg | New |
| #46 | 2119 | T→C | 707 | Ser→Pro | New |
| #47 | 2119 | T→C | 707 | Ser→Pro | New |
| #52 | 1810 | C→T | 604 | Pro→Ser | New |
| | 4388 | T→G | | Phe→Cys | |
| #54 | 146 | C→G | 49 | Ser→Cys | Vorechovsky, I. et al., 1996 |
| #55 | 6096 | A→T | | Arg→Ser | New |
| #57 | 4258 | C→T | 1420 | Leu→Phe | Vorechovsky, I. et al, 1996 |
| #61 | 5557 | G→A | 1853 | Asp→Asn | Sandoval, N et al., 1999 |
| #63 | 5557 | G→A | 1853 | Asp→Asn | Sandoval, N. et al., 1999 |
| #64 | 378 | T→A | 126 | Asp→Glu | New |

Comments
1) 12 out of 63 controls (19%) were identified as carriers of known mutations
2) Control #2 is the mother of an HD patient
3) Mutation at position 5557 was found in 8 healthy controls

TABLE 6
Predominant sequence variations in BC-BC vs. healthy controls

| | | | | | | Amino acid |
|---|---|---|---|---|---|---|
| | | BC- | Healthy | % | % Healthy | change in |
| No. | Mutation | BC | controls | BC-BC | controls | protein |
| 1 | 3161 C→G | 5/70 | 1/63 | 7.1% | 1.6% | Pro→Arg |
| 2 | 2572 T→C | 3/70 | 0/63 | 4.3% | 0.0% | Phe→Leu |
| 3 | 6235 G→A | 3/70 | 0/63 | 4.3% | 0.0% | Met→Val |
| 4 | 3118 A→G | 2/70 | 0/63 | 2.9% | 0.0% | Val→Ile |
| 5 | 378 T→A | 2/70 | 2/63 | 2.9% | 3.2% | |

The frequency of carriers of all these mutations is 15/70=21.4% among all BC-BC patients, whereas the frequency in healthy controls is 3/63=4.8%.

Two combinations are unique to BC-BC patients: (i) position 3161 (C→G)+position 2572(T→C) (3/70); and (ii) position 6235(G→A)+position 378(T→A) (2/70), representing a total of 5/70=7% in BC-BC patients as compared to 0/63=0% in normal healthy controls.

Sixteen (16) mutations were identified, 9 of which were in the BC-BC patient cohort (Table 4) and 7 more of which were found in the healthy control cohort (Table 5) (above and in PCT Patent Application WO 01/68668). These new mutations are linked to a predisposition to cancer in males and females, particularly to breast cancer.

Note that the total number of carriers among the BC-BC patients is 28/70, or 40%, whereas the total number of carriers among healthy controls is 18/63, or 29%. Regarding the mutation at position 5557, which is probably a polymorphism, the total number of carriers among the BC-BC patients is 14/70, or 20%, whereas the total number of carriers among the healthy control cohort is 8/63, or 13%. Almost all (98%, corresponding to 43/44) of the sequence variations identified in this study were missense mutations (point mutations), i.e., substitution of the wild-type amino acid residue with an abnormal residue. This pattern is markedly different from that reported for A-T patients, in which the predominant sequence variations lead to protein truncation.

The identified variations in the ATM sequence in the present study are distributed evenly along most of its ORF, but none of the sequence variations were found within the PI-3 kinase domain in the carboxy terminal region of the gene. It is likely that mutations located in the catalytic site of the PI-3 kinase would cause severe phenotypes such as A-T.

Mutations identified in healthy controls do not display localization preference and all of them occur with an almost equal, and low, frequency.

### CONCLUSION

Generally, three groups of mutations in the ATM gene were found, which are as follows:

1) occurs predominantly in primary BC: position 146 (C→G), and position 1636 (C→G),

2) occur in primary BC and BC-BC at comparable frequencies: position 378 (T→A), position 2572 (T→C), position 2614 (C→T), position 3118 (A→G), and position 3161 (C→G), and

3) occurs predominantly in BC-BC: position 6235 (G→A). The mutation at position 378 (T→A) appears in BC-BC only in combination with position 6235 (G→A). In healthy controls the mutation at position 378 (T→A) does not appear in combination with position 6235 (G→A).

There is a significant correlation between breast cancer and the specific sequence variations disclosed herein The mutations found are significant in the diagnosis of predisposition to cancer, particularly breast cancer (a cancer of epithelial origin).

### Example 2

### Correlation of the Mutations with Other Cancers

The point mutations disclosed herein, in Table 3 and Table 4, are predictive of cancers other than breast cancer. In particular they are predictive of an epithelial-derived cancer (a cancer of epithelial origin) other than breast cancer, such as lung cancer, colon cancer, prostate cancer, ovarian cancer, bladder cancer, and cancer of the pancreas, and also of a lymphoproliferative malignancy, such as HD and non-Hodgkin's lymphoma.

To show this experimentally, we screen patient-derived samples, preferably blood, for the presence of these mutations.

We find that the frequency of one or more of the ATM point mutations occurs at a higher level in patient-derived samples (wherein the patient has a particular cancer) compared to samples from healthy individuals (controls). These point mutations are thus predictive of the specific cancer. Mutations in healthy controls predominantly occur at an almost equal, and low, frequency.

The various patient-derived materials may be tissue samples or blood, preferably blood, which are then examined by methods known in the art for the presence of the mutations. These methods are more fully described in Example 1. Such methods include, but are not limited to, the methods discussed below. Note that there are many methods known in the art for testing genomic DNA and cDNA for mutations, including point mutations, as described in this specification. Methods that can be used for testing genomic DNA require use of the primers described in the specification. DNA methods that are used can include, but are not limited to, the following inter alia:

a. polymerase chain reaction (POR)-restriction enzyme assay (Sueoka, H et al., 2000),

b. PCR and LightCycler technology (Funayo, T et al., 2000; Pais, G et al., 2001),

c. allele-specific PCR (MacLeod, S L et al., 2000),

d. restriction enzyme digestion (Ho, L L et al., 2001),

e. denaturing high performance liquid chromatography (dHPLC), for fast and sensitive analysis of PCR-amplified DNA fragments (Oldenburg, J et al., 2001),

f. restriction endonuclease fingerprinting single-strand conformation polymorphism (REF-SSCP) (Jugessur, A et al., 2000; Liu, Q et al., 1995), and

g. detection of single base substitutions as heteroduplex polymorphims (White, B. M et al., 1991).

Complete sequencing of the cDNA of the ATM gene in the above patient-derived materials is also performed, as described in Example 1, and new predictive mutations for these cancers are found. These new predictive mutations and their use in screening for, and diagnosis of, specific cancers are considered part of the instant invention.

### Example 3

### Screening Assays for Mutations in DNA

This invention is directed to germline mutations in the ATM gene which, when present in an individual, lead to a greater risk of the individual developing a cancer, in particular, a cancer of epithelial origin such as lung cancer, colon cancer, prostate cancer, ovarian cancer, bladder cancer, and cancer of the pancreas, and also a lymphoproliferative malignancy such as HD and non-Hodgkin's lymphoma.

The methods of the present invention provide that either presumptively healthy men and women and/or men and women known to be at risk for cancer are screened by obtaining various patient-derived materials such as tissue samples or blood, preferably blood, which are then examined by methods known in the art for the presence of the mutation or mutations. These methods are more fully described in Example 1. Such methods include, but are not limited to, the methods discussed below. Note that there are many methods known in the art for testing genomic DNA and cDNA for mutations, including point mutations, as described in this specification. Some of the methods that can be used for testing genomic or other DNA require use of oligonucleotides such as the primers described in this specification, or based on these primers. Such oligonucleotides are termed probes, and each such probe is designed to be capable of detecting at least one specific mutation in the open reading frame of the ATM gene (SEQ.ID.NO:1) in a DNA sample, which mutation is selected from the group consisting of the mutations set forth in Table 3 and Table 4. Various hybridization techniques may be used in detecting mutations and such methods are well-known in the art. For example, hybridization may be performed by measurement of hybridization of the probe to the DNA sample under varying conditions of stringency. Said conditions comprise hybridization as well as washing conditions. By setting the stringency conditions, a person skilled in the art can determine if the probe is exactly complementary to the sample DNA or not.

The choice of conditions is within the skill of one in the art. Such conditions can be determined according to protocols described, for example, in Sambrook, Molecular Cloning, A Laboratory Manual, 2nd edition (1989), Cold Spring Harbor Laboratory Press, Cold Spring Harbor, N.Y. or Hames and Higgins, “Nucleic acid hybridization, a practical approach”, IRL Press, Oxford(1985).

DNA methods that are used can include, but are not limited to, the following inter alia:

a. polymerase chain reaction (PCR)-restriction enzyme assay (Sueoka, H et al., 2000),

b. PCR and LightCycler technology (Funayo, T et al., 2000; Pais, G et al., 2001),

c. allele-specific PCR (MacLeod, S L et al., 2000),

d. restriction enzyme digestion (Ho, L L et al., 2001),

e. denaturing high performance liquid chromatography (dHPLC), for fast and sensitive analysis of PCR-amplified DNA fragments (Oldenburg, J et al., 2001),

f. restriction endonuclease fingerprinting single-strand conformation polymorphism (REF-SSCP) (Jugessur, A et al., 2000; Liu, Q et al., 1995), and

g. detection of single base substitutions as heteroduplex polymorphims (White, B M et al., 1991).

Routine screening of DNA from biological samples for various genetic conditions which are diseases caused by mutations is well known in the art. This has been accomplished for the following diseases inter alia: phenylketonuria (PKU) (Sueoka, H et al., 2000), APRT deficiency (Funayo, T et al., 2000), X-linked thrombocytopenia (XLT) (Ho, L L et al., 2001), hemophilia A (Oldenburg, J et al., 2001), cystic fibrosis (CF) (Mastella, G et al., 2001), Gaucher's disease (Kronn D et al., 1998), fragile-X syndrome (Toledano-Alhadef H et al., 2001), and Canavan disease (Matalon, R et al., 1995). Similar methods are used in the subject invention to screen patients for the presence of the various mutations disclosed.

Throughout this application various publications are referenced by author and year. Full citations for the publications are listed below. The disclosures of these publications in their entireties are hereby incorporated by reference into this application in order to more fully describe the state of the art to which this invention pertains.

The invention has been described in an illustrative manner, and it is to be understood that the terminology that has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

### REFERENCES

Aisenberg, A C et al., (1997), J. Am. Cancer Soc., pp. 1203-1209

Easton, D F (1994) Int. J. Rad. Biol., Vol. 66, pp. S177-S182

Funayo, T et al., (2000) J Clin.Lab.Anal., Vol.14(6), pp. 274-279

Hancock, S L, Tucker, M A and Hoppe, R T (1993) J. Natl. Cancer Inst., Vol. 85, pp. 25-31

Ho, L L et al., (2001) Br. J. Haematol., Vol. 112(1), pp. 76-80

Izatt L, et al. (2000) in. Genes Chromosomes Cancer, Vol. 26, pp. 286-294

Jugessur, A. et al., (2000) J.Mol.Med., Vol. 78 (10), pp. 580-587

Kronn D et al., (1998) Arch Intern Med, Vol. 158(7), pp. 777-781

Liu, Q et al., (1995) Biotechniques, Vol. 18, pp.470-477

MacLeod, S L et al., (2000) Ann Surg. Oncol., Vol. 7(10), pp. 777-782

Mastella, G. et al., (2001) Pancreatology, Vol. 1(5), pp. 531-537

Matalon, R et al., (1995) J Inherit Metab Dis, Vol. 18(2), pp. 215-217

Meyn, M S (1999) Clin. Genetics, Vol. 55, pp. 289-304

Morrell, D, et al., (1990) Cancer Genet. Cytogenet. Vol. 50, pp. 119-123

Oldenburg, J et al., (2001) J. Biochem. Biophys. Methods, Vol. 47(1-2), pp. 39-51

Pais, G et al., (2001) J. Biochem. Biophys. Methods, Vol. 47(1-2), pp. 121-129

Sandoval, N et al., (1999) Hum Mol. Gen., Vol. 8, pp. 69-79

Savitsky, K et al., (1995) Hum. Mol. Genet., Vol. 4, pp. 2025-2032

Savitsky, K et al., (1995) Science, Vol. 268, pp. 1749-1753

Savitsky, K et al., (1997) Nucleic Acids Research Vol. 25(9), pp. 1678-1684

Sueoka, H et al (2000) Genet Test Vol. 4(3), pp. 249-256

Swift, M et al., (1987) New Engl. J. Med., Vol. 316, pp. 1289-1294

Swift, M, et al., (1991) New Engl. J. Med., Vol. 325, pp. 1831-1836

Telatar, M et al., (1996) Am. J. Hum. Genet, Vol 59, pp. 40-44

Toledano-Alhadef H et al., (2001) Am. J. Hum Genet, Vol 69(2), pp. 351-360

Uziel, T et al., (1996) Genomics, Vol. 33, pp. 317-320

Vorechovsky, I et al., (1996) Can. Res., Vol. 56, pp. 2726 - 2732

Vorechovsky, I et al. (1997) Nat. Genet Vol. 17, pp.96-97

White, B M et al., (1991) Genomics, Vol. 12, pp. 301-306

Yahalom, J et al., (1992) J. Clin. Oncol., Vol 10(11), pp. 1674-1681

What is claimed is:

1. A method of testing a subject to determine if the subject has a predisposition for developing a cancer which comprises detecting at least one mutation in the open reading frame of the ATM gene (SEQ.ID.NO:1) in a DNA sample from the subject, which mutation is selected from the group consisting of the mutations set forth in Table 3 and Table 4;
wherein the cancer is selected from the set of cancers consisting of lung cancer, colon cancer, prostate cancer, ovarian cancer, bladder cancer, cancer of the pancreas, Hodgkin's disease and non-Hodgkin's lymphoma;
and wherein the presence of such a mutation indicates that the subject has a predisposition for developing such a cancer.

2. The method according to claim 1, wherein the mutation is selected from the group consisting of 3161 C→G, 2572 T→C, 6235 G→A, 3118 A→G, 378 T→A, 2614 C→T, 146 C→G, and 1636 C→G.

3. The method according to claim 1, wherein the mutation is selected from the group consisting of a double mutation 3161 (C→G) and 2572 (T→C), and a double mutation 6253 (G→A) and 378 (T→A).

4. The method of claim 1 wherein the DNA is cDNA.

5. The method of claim 1 wherein the DNA is genomic DNA.

6. The method of any one of claims 1-5 wherein the cancer is an epithelial-derived cancer, wherein the cancer is selected from the set consisting of lung cancer, colon cancer, prostate cancer, ovarian cancer, bladder cancer, and cancer of the pancreas.

7. The method of claims 1-5 wherein the cancer is selected from the set of cancers consisting of Hodgkin's disease and non-Hodgkin's lymphoma.

8. The method of claim 6 wherein the cancer is lung cancer.

9. The method of claim 6 wherein the cancer is colon cancer.

10. The method of claim 6 wherein the cancer isprostate cancer.

11. The method of claim 6 wherein the cancer is ovarian cancer.

12. The method of claim 6 wherein the cancer is bladder cancer.

13. The method of claim 6 wherein the cancer is cancer of the pancreas.

14. The method of claim 7 wherein the cancer is Hodgkin's disease.

15. The method of claim 7 wherein the cancer is non-Hodgkin's lymphoma.

16. An oligonucleotide probe which is capable of detecting a mutation in the open reading frame of the ATM gene (SEQ.ID.NO:1) in a DNA sample, which mutation is selected from the group consisting of the mutations set forth in Table 3 and Table 4.

17. The probe according to claim 16, wherein said mutation is selected from the group consisting of 378 T→A, 3383 A→G, 1636 C→G, 2614 C→T, 6437 G→C, 2932 T→C, 2289 T→A, 6096 A→T, 6176 C→T, 6919 C→T, 3925 G→A, 6067 G→A, 2119 T→C, 1810 C→T, and 4388 T→G
